(19) United States
(12) Patent Application Publication
(10) Pub. No.: US 2025/0255583 A1
(43) Pub. Date: Aug. 14, 2025

(54) ULTRASONIC IMAGING METHOD AND ULTRASONIC DEVICE

(71) Applicant: SHENZHEN MINDRAY BIO-MEDICAL ELECTRONICS CO. LTD., Shenzhen (CN)

(72) Inventors: Wenhui CHEN, Shenzhen (CN); Chaoyue Liu, Shenzhen (CN); Yaoxian Zou, Shenzhen (CN); Muqing Lin, Shenzhen (CN)

(21) Appl. No.: 19/195,402

(22) Filed: Apr. 30, 2025

Related U.S. Application Data
(63) Continuation of application No. PCT/CN2023/127760, filed on Oct. 30, 2023.

(30) Foreign Application Priority Data
Oct. 31, 2022 (CN) 202211352167.5

(51) Int. Cl. A61B 8/00; A61B 8/08; G06T 7/00; G06T 15/00; G06T 15/60

(52) U.S. Cl. CPC A61B 8/5215; A61B 8/085; A61B 8/466; A61B 8/5207; A61B 8/54; G06T 7/0012; G06T 15/005; G06T 15/60; G06T 2207/10136; G06T 2207/20; G06T 2207/30004

(57) ABSTRACT

The present disclosure provides an ultrasonic imaging method. The method comprises the following steps: obtaining a three-dimensional data to be rendered of an ovary and multiple follicles wrapped in the ovary of an examined object; detecting an ovary region corresponding to the ovary and multiple follicle regions corresponding to the multiple follicles in the three-dimensional data; determining transparency coefficients of different portions of the ovary region according to results of the detection; and rendering the three-dimensional data according to the determined transparency coefficients to obtain a rendered image of the ovary region and the multiple follicle regions. In the rendered image, different portions of the ovarian region exhibit different transparencies.

### CROSS-REFERENCE TO RELATED APPLICATIONS

This application is a Continuation Application of International Patent Application No. PCT/CN2023/127760, filed on Oct. 30, 2023, which is based on and claims priority to and benefits of Chinese Patent Application No. 202211352167.5, filed on Oct. 31, 2022. The entire content of all of the above-referenced applications is incorporated herein by reference.

### TECHNICAL FIELD

The present disclosure relates to medical devices, and more particularly, to ultrasonic imaging methods and ultrasonic devices.

### BACKGROUND

There is a wrapping or obstructing relationship between many organs or tissues in the human body, such as ovaries and follicles, uterus and endometrium, etc. Therefore, when image data of these organs are three-dimensional rendered and displayed, the image data of the occluded organs will not be visible. Therefore, transparent rendering of the image data of the outer organ can more clearly show the shape and structure of the occluded organs, as well as the positional relationship of the two organs, etc., so as to better assist doctors in clinical diagnosis. For example, three-dimensional hybrid rendering of the image data of ovaries and follicles can assist doctors in ovarian receptivity analysis, and three-dimensional hybrid rendering of the image data of cervix and uterine body can enable doctors to judge the degree of tilt of the uterus according to the positional relationship between the rendered cervix and uterine body.

In the current hybrid rendering, a fixed transparency coefficient is set for the organs that need to be displayed transparently, by which the three-dimensional sense of the rendered organ is insufficient, the transformation of the organs at different depths or angles cannot be reflected, the visual experience is poor, and the positional relationship between the organs presented is also not clear enough, which may affect doctor's clinical diagnosis.

### SUMMARY

A series of concepts in simplified form are introduced in the SUMMARY, which will be described in further detail in the DETAILED DESCRIPTION. The SUMMARY of the present disclosure does not intend to define the key features and essential technical features of the claimed technical solutions, nor intend to determine the protection scope of the claimed technical solutions.

In an embodiment, an ultrasonic imaging method is provided, which may include:
obtaining a three-dimensional data to be rendered of an ovary and multiple follicles wrapped in the ovary of an examined object;
detecting an ovary region corresponding to the ovary and multiple follicle regions corresponding to the multiple follicles in the three-dimensional data to obtain results of the detection;
determining transparency coefficients of different portions of the ovary region according to the results of the detection; and
rendering the three-dimensional data according to the determined transparency coefficients to obtain a rendered image of the ovary region and the multiple follicle regions, where, in the rendered image, the different portions of the ovary region are represented with different transparency.

In an embodiment, the method may further include:
determining anterior portions of the ovary region from a user's perspective according to the results of the detection, where
determining the transparency coefficients of different portions of the ovary region may include: determining the transparency coefficients of the anterior portions and other portions of the ovary region, respectively; and
in the rendered image, the anterior portions of the ovary region are represented with a variety of different transparency, and the other portions are represented with a same transparency or opacity.

In an embodiment, the method may further include:
determining target portions to be rendered in the ovary region; where
determining the transparency coefficients of different portions of the ovary region may include: determining the transparency coefficients of the target portions and the other portions of the ovary region, respectively; and
in the rendered image, the target portions of the ovary region are represented with a variety of different transparency, and the other portions are represented with a same transparency or opacity.

In an embodiment, the method may further include:
determining colors for rendering the ovary region and the multiple follicle regions; and
rendering the three-dimensional data according to the determined colors.

In an embodiment, the method may further include:
determining a target follicle region that obscures one or more other follicle regions under a user's perspective from the multiple follicle regions according to the results of the detection;
determining transparency coefficients of different portions of the target follicle region; where,
in the rendered image, the different portions of the target follicle region are represented with different transparency.

In an embodiment, rendering the three-dimensional data may include rendering the three-dimensional data with a surface rendering method, and determining the transparency coefficients of different portions of the ovary region according to the results of the detection may include:
obtaining vertex coordinates of a mesh model of the ovary region from the three-dimensional data according to the results of the detection, where the vertex coordinates are used to form a plurality of triangular sheets for constructing the mesh model of the ovary region; and
for at least a part of the plurality of triangular sheets, performing following steps:
calculating a normal vector of the triangular sheet according to the coordinates of three vertices of the triangular sheet;
calculating an angle between the normal vector of the triangular sheet and a preset reference direction; and
determining a transparency coefficient of the triangular sheet according to a size of the angle.

In an embodiment, rendering the three-dimensional data may include rendering the three-dimensional data with a surface rendering method, and determining the transparency coefficients of different portions of the ovary region according to the results of the detection may include:
obtaining vertex coordinates of a mesh model of the ovary region and a coordinate of center of gravity of the ovary region from the three-dimensional data according to the results of the detection, where the vertex coordinates are used to form a plurality of triangular sheets for constructing the mesh model of the ovary region; and
for at least a part of the plurality of triangular sheets, performing following steps:
calculating direction vectors between three vertices of the triangular sheet and the center of gravity of the mesh model according to the coordinates of the three vertices of the triangular sheet;
calculating angles between the direction vectors and a preset reference direction; and
determining a transparency coefficient of the triangular sheet according to sizes of the angles.

In an embodiment, rendering the three-dimensional data may include rendering the three-dimensional data with a surface rendering method, and determining the transparency coefficients of different portions of the ovary region according to the results of the detection may include:
obtaining vertex coordinates of a mesh model of the ovary region from the three-dimensional data according to the results of the detection, where the vertex coordinates are used to form a plurality of triangular sheets for constructing the mesh model of the ovary region; and
for at least a part of the plurality of triangular sheets, performing following steps:
calculating angles between three vertices of the triangular sheet and a preset reference direction according to the coordinates of the three vertices of the triangular sheet; and
determining a transparency coefficient of the triangular sheet according to sizes of the angles.

In an embodiment, rendering the three-dimensional data may include rendering the three-dimensional data with a volume rendering method, and determining the transparency coefficients of different portions of the ovary region according to the results of the detection may include:
determining a contour of the ovary region according to the results of the detection; and
determining transparency coefficients of at least a part of pixels within the contour of the ovary region corresponding to the volume rendering method.

In an embodiment, rendering the three-dimensional data may include rendering the three-dimensional data with a volume rendering method, and determining the transparency coefficients of different portions of the ovary region according to the results of the detection may include:
determining a contour of the ovary region according to the results of the detection;
obtaining gray values of at least a part of pixels within the contour of the ovary region from the three-dimensional data; and
determining transparency coefficients of the at least a part of pixels according to a preset mapping relationship between gray values and transparency coefficients.

In an embodiment, detecting the ovary region corresponding to the ovary and the multiple follicle regions corresponding to the multiple follicles in the three-dimensional data may include:
detecting the ovary region and the multiple follicle regions in the three-dimensional data with an object detection method; or
detecting the ovary region and the multiple follicle regions in the three-dimensional data with an object segmentation method; or
detecting a part of the ovary region and the multiple follicle regions in the three-dimensional data with an object detection method, and detecting the other part of the ovary region and the multiple follicle regions in the three-dimensional data with an object segmentation method.

In an embodiment, obtaining the three-dimensional data to be rendered of the ovary and the multiple follicles wrapped in the ovary of the examined object may include:
obtaining a four-dimensional ultrasonic image of the ovary and the multiple follicles of the examined object; and
selecting a three-dimensional ultrasonic image at a moment from the four-dimensional ultrasonic image as the three-dimensional data to be rendered according to one of an object recognition method, an object detection method and an object segmentation method.

In an embodiment, an ultrasonic imaging method is provided, which may include:
obtaining a three-dimensional data to be rendered of a first organ and a second organ of an examined object, where the first organ at least partially wraps the second organ;
detecting a first organ region corresponding to the first organ and a second organ region corresponding to the second organ in the three-dimensional data to obtain results of the detection;
determining transparency coefficients of different portions of the first organ region according to the results of the detection; and
rendering the three-dimensional data according to the determined transparency coefficients to obtain a rendered image of the first organ region and the second organ region, where, in the rendered image, different portions of the first organ region are represented with different transparency.

In an embodiment, the method may further include:
determining anterior portions of the first organ region from a user's perspective according to the results of the detection, where
determining the transparency coefficients of different portions of the first organ region may include: determining the transparency coefficients of the anterior portions and other portions of the first organ region, respectively; and
in the rendered image, the anterior portions of the first organ region are represented with a variety of different transparency, and the other portions are represented with a same transparency or opacity.

In an embodiment, the method may further include:
determining target portions to be rendered in the first organ region; where
determining the transparency coefficients of different portions of the first organ region may include: determining the transparency coefficients of the target portions and the other portions of the ovary region, respectively; and
in the rendered image, the target portions of the first organ region are represented with a variety of different transparency, and the other portions are represented with a same transparency or opacity.

In an embodiment, the method may further include:
determining colors for rendering the first organ region and the second organ region; and
rendering the three-dimensional data according to the determined colors.

In an embodiment, rendering the three-dimensional data may include rendering the three-dimensional data with a surface rendering method, and determining the transparency coefficients of different portions of the first organ region according to the results of the detection may include:
obtaining vertex coordinates of a mesh model of the first organ region from the three-dimensional data according to the results of the detection, where the vertex coordinates are used to form a plurality of triangular sheets for constructing the mesh model of the first organ region; and
for at least a part of the plurality of triangular sheets, performing following steps:
calculating a normal vector of the triangular sheet according to the coordinates of three vertices of the triangular sheet;
calculating an angle between the normal vector of the triangular sheet and a preset reference direction; and
determining a transparency coefficient of the triangular sheet according to a size of the angle.

In an embodiment, rendering the three-dimensional data may include rendering the three-dimensional data with a surface rendering method, and determining the transparency coefficients of different portions of the first organ region according to the results of the detection may include:
obtaining vertex coordinates of a mesh model of the first organ region and a coordinate of center of gravity of the first organ region from the three-dimensional data according to the results of the detection, where the vertex coordinates are used to form a plurality of triangular sheets for constructing the mesh model of the first organ region; and
for at least a part of the plurality of triangular sheets, performing following steps:
calculating direction vectors between three vertices of the triangular sheet and the center of gravity of the mesh model according to the coordinates of the three vertices of the triangular sheet;
calculating angles between the direction vectors and a preset reference direction; and
determining a transparency coefficient of the triangular sheet according to sizes of the angles.

In an embodiment, rendering the three-dimensional data may include rendering the three-dimensional data with a surface rendering method, and determining the transparency coefficients of different portions of the first organ region according to the results of the detection may include:
obtaining vertex coordinates of a mesh model of the first organ region from the three-dimensional data according to the results of the detection, where the vertex coordinates are used to form a plurality of triangular sheets for constructing the mesh model of the first organ region; and
for at least a part of the plurality of triangular sheets, performing following steps:
calculating angles between three vertices of the triangular sheet and a preset reference direction according to the coordinates of the three vertices of the triangular sheet; and
determining a transparency coefficient of the triangular sheet according to sizes of the angles.

In an embodiment, rendering the three-dimensional data may include rendering the three-dimensional data with a volume rendering method, and determining the transparency coefficients of different portions of the first organ region according to the results of the detection may include:
determining a contour of the first organ region according to the results of the detection; and
determining transparency coefficients of at least a part of pixels within the contour of the first organ region corresponding to the volume rendering method.

In an embodiment, rendering the three-dimensional data may include rendering the three-dimensional data with a volume rendering method, and determining the transparency coefficients of different portions of the first organ region according to the results of the detection may include:
determining a contour of the first organ region according to the results of the detection;
obtaining gray values of at least apart of pixels within the contour of the first organ region from the three-dimensional data; and
determining transparency coefficients of the at least a part of pixels according to a preset mapping relationship between gray values and transparency coefficients.

In an embodiment, detecting the first organ region and the second region in the three-dimensional data may include:
detecting the first organ region and the second region in the three-dimensional data with an object detection method; or
detecting the first organ region and the second region in the three-dimensional data with an object segmentation method; or
detecting one of the first organ region and the second region in the three-dimensional data with an object detection method, and detecting the other of the first organ region and the second region in the three-dimensional data with an object segmentation method.

In an embodiment, obtaining the three-dimensional data to be rendered of the first organ and the second organ of the examined object may include:
obtaining a four-dimensional ultrasonic image of the first organ and the second organ of the examined object; and
selecting a three-dimensional ultrasonic image at a moment from the four-dimensional ultrasonic image as the three-dimensional data to be rendered according to one of an object recognition method, an object detection method and an object segmentation method.

In an embodiment, the first organ is an ovary and the second organ is a follicle; or, the first organ is an uterine body and the second organ is an endometrium; or, the first organ is a fetal brain and the second organ is an internal anatomical structure of the fetal brain; or, the first organ is a fetal abdomen and the second organ is an internal anatomical structure of the fetal abdomen; or, the first organ is a liver and the second organ is an intrahepatic blood vessel; or, the first organ is a heart and the second organ is an internal structure of the heart.

In an embodiment, an ultrasonic device is provided, which may include:
an ultrasonic probe;
a transmitting/receiving circuit configured to control the ultrasonic probe to transmit ultrasonic waves to an ovary of an object to be examined, and to control the ultrasonic probe to receive echo signals of the ultrasonic waves;
a memory configured to store computer-executable instructions;
a processor configured to execute the computer-executable instructions to obtain three-dimensional data according to the echo signals and perform an ultrasonic imaging method of any one of the embodiments above to generate a rendered image; and
a display configured to display the rendered image.

In an embodiment, an ultrasonic device is provided, which may include:
an ultrasonic probe;
a transmitting/receiving circuit configured to control the ultrasonic probe to transmit ultrasonic waves to a target tissue of an object to be examined, and to control the ultrasonic probe to receive echo signals of the ultrasonic waves;
a memory configured to store computer-executable instructions;
a processor configured to execute the computer-executable instructions to obtain three-dimensional data according to the echo signals and perform an ultrasonic imaging method of any one of the embodiments above to generate a rendered image; and
a display configured to display the rendered image.

According to the present disclosure, when it is desired to render an organ that is wrapped or obscured, the transparency coefficients of different parts of the outer organ can be determined, such that different parts of the outer organ are presented in different transparency, thereby increasing the three-dimensional sense of the outer organ. Therefore, a rendering effect with clear positional relationships can be presented to the user, such that the user can clearly see the position, size, and inclusion relationship between the organs, etc., which enhances the visual experience and helps to improve work efficiency and accuracy.

### BRIEF DESCRIPTION OF THE DRAWINGS

In order to more clearly illustrate the technical solutions in the embodiments of the present disclosure, the drawings that will be used in the description of the embodiments will be briefly introduced below. Obviously, the drawings described below are merely some embodiments of the present disclosure. For those of ordinary skill in the art, other drawings can be obtained from these drawings without creative effort.

In the drawings:

FIG. 1 shows a schematic block diagram of an ultrasonic device according to an embodiment of the present disclosure;

FIG. 2 shows a schematic flowchart of an ultrasonic imaging method according to an embodiment of the present disclosure;

FIG. 3a to FIG. 3c show schematic diagrams of rendered images according to embodiments of the present disclosure;

FIG. 4 shows a schematic flowchart of an ultrasonic imaging method according to another embodiment of the present disclosure; and

FIG. 5 shows a schematic diagram of a computing device according to an embodiment of the present disclosure.

### DETAILED DESCRIPTION

In order to make the objectives, technical solutions and advantages of the present disclosure more apparent, exemplary embodiments of the present disclosure will be described in detail below with reference to the drawings. However, the described embodiments are only part, but not all, of the embodiments of the present disclosure. It should be understood that the present disclosure is not limited by the embodiments described herein. Based on the embodiments of the present disclosure described herein, all other embodiments obtained by those skilled in the art without creative effort should fall within the protection scope of the present disclosure.

In the description below, numerous specific details are given in order to provide a more thorough understanding to the present disclosure. However, it will be apparent to those skilled in the art that the present disclosure may be implemented without one or more of these details. In other examples, in order to avoid confusion with the present disclosure, some technical features known in the art will not been described.

It should be understood that the present disclosure can be implemented in different forms and should not be construed as being limited to the embodiments described herein. On the contrary, the provision of these embodiments will enable the disclosure to be thorough and complete, and fully convey the scope of the present disclosure to those skilled in the art.

The terminology used herein is for the purpose of describing the specific embodiments only, but not intended to be a limitation to the present disclosure. As used herein, the singular forms “a,” “an,” and “the/said” are also intended to include the plural forms, unless the context clearly indicates otherwise. It should also be understood that the terms “form” and/or “comprise” used herein represent the presence of said feature, integer, step, operation, element, and/or component, but not preclude the presence or addition of one or more other features, integers, steps, operations, elements, components and/or groups. As used herein, the term “and/or” means any and all combinations of related listed items.

For a thorough understanding to the present disclosure, detailed structures will be described below so as to illustrate the technical solutions proposed by the present disclosure. Alternative embodiments of the present disclosure will be described in detail below. However, in addition to these detailed descriptions, the present disclosure may also have other embodiments.

The present disclosure provides ultrasonic imaging methods and ultrasonic apparatuses. When it is desired to render organs between which there is a wrapping or obstructing relationship, the transparency coefficient corresponding to different parts of the outer organ can be determined, such that different parts of the outer organ can be represented with different transparencies, thereby increasing the three-dimensional sense of the outer organ. Accordingly, it is possible to present a clear rendering effect of the positional relationship to the user, such that the user can clearly see the position, size and inclusion relationship, etc. between the organs, thereby improving the visual experience and increasing the accuracy of diagnostic analysis.

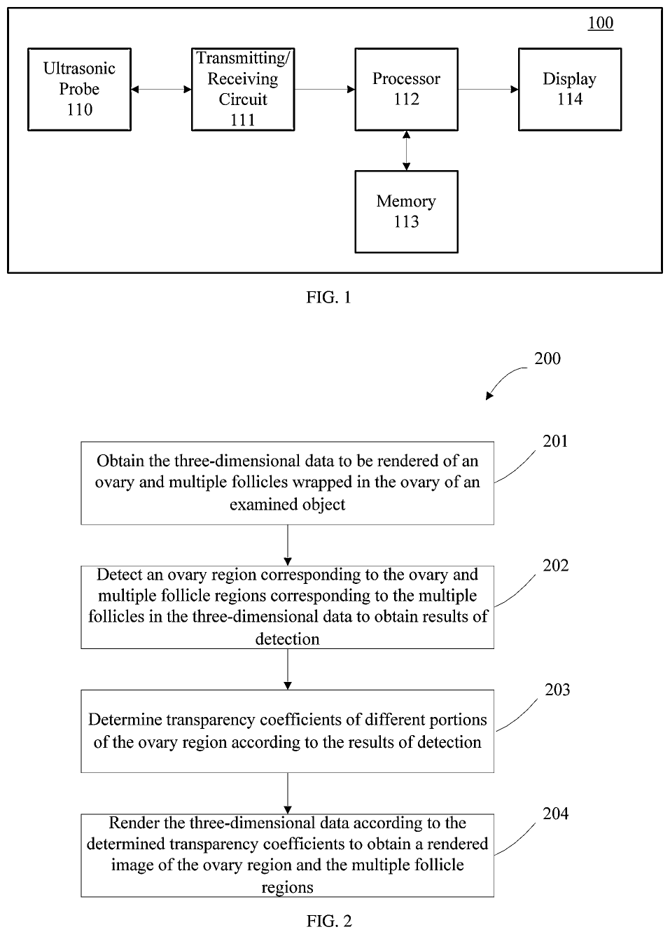

An ultrasonic apparatus according to an embodiment of the present disclosure will be described with reference to FIG. 1 that shows a schematic block diagram of the ultrasonic apparatus according to an embodiment of the present disclosure.

As shown in FIG. 1, the ultrasonic apparatus 100 includes an ultrasonic probe 110, a transmitting/receiving circuit 111, a processor 112, a memory 113 and a display 114. The ultrasonic apparatus 100 may further include a beam forming circuit and a transmitting/receiving switch, etc.

The ultrasonic probe 110 may include an array of multiple elements. During each transmitting of ultrasonic waves, all or some of the elements of the ultrasonic probe 110 participate in the transmitting of ultrasonic waves. In this case, each element or each part of the elements participating in the ultrasonic transmitting is excited by the transmitting pulse to transmit ultrasonic waves respectively. The ultrasonic waves transmitted by these elements are superimposed during propagation to form a synthetic ultrasonic beam that is transmitted to the area of the object being examined where the region of interest is located. For example, the region of interest may be the ovarian area, the uterine area, etc.

The transmitting/receiving circuit 111 may be coupled to the ultrasonic probe 110 through the transmitting/receiving switch. The transmitting/receiving switch may also be referred to as a transmitting/receiving controller, which may include a transmitting controller and a receiving controller. The transmitting controller may be configured to excite the ultrasonic probe 110 to transmit ultrasonic waves to the region of interest of the object to be examined by the transmitting circuit. The receiving controller may be configured to receive ultrasonic echoes returned from the region of interest of the object to be examined through the ultrasonic probe 110 by the receiving circuit, thereby obtaining the echo signals of the ultrasonic waves. Thereafter, the transmitting/receiving circuit 111 sends the echo signals to the beam forming circuit. The beam forming circuit may perform processing such as focus delay, weighting and channel summation, etc. on the echo signals, and then send the processed ultrasonic echo data to the processor 112.

The processor 112 may be implemented in software, hardware, firmware, or any combination thereof. The processor 112 may use circuit, single or multiple application specific integrated circuits (ASICs), single or multiple general purpose integrated circuits, single or multiple microprocessors, single or multiple programmable logic devices, or any combination of the foregoing circuits or devices, or other suitable circuits or devices, such that the processor 112 can perform the respective steps of the methods in various embodiments of the present disclosure. The processor 112 may control other components in the ultrasonic apparatus 100 to perform desired functions.

The processor 112 may process the received echo signals of the ultrasonic waves to obtain a three-dimensional ultrasonic image of the region of interest of the object being examined. In this process, the ultrasonic probe 110 may transmit the ultrasonic waves or receive the echoes in a series of scanning planes. The processor 112 may process the echo signals according to the three-dimensional spatial relationship of the scanning planes, so as to realize the scanning of the region of interest of the object being examined in three-dimensional space and the reconstruction of the three-dimensional image. The processor 112 may perform some or all of the image post-processing steps such as denoising, smoothing and enhancement, etc., thereby obtaining the three-dimensional ultrasonic image of the region of interest of the object being examined. The obtained three-dimensional ultrasonic image may be stored in the memory 113, be displayed on the display 114, or be transmitted to other storage devices for storage via wired or wireless communication lines.

The memory 113 may be configured to store instructions to be executed by the processor, store received echo signals of ultrasonic waves, store ultrasonic images, etc. The memory may be a flash memory card, a solid state memory, a hard disk, etc. It may be volatile memory and/or non-volatile memory. It may be removable memory and/or non-removable memory, etc.

The display 114 is communicatively coupled with the processor 112. The display 114 may be a touch screen display, a liquid crystal display, or the like. Although in the present embodiment, the display 114 is shown as being part of the ultrasonic apparatus 100, in other embodiments, the display 114 may be a separate display device such as a liquid crystal display or a television, etc. that is independent of the ultrasonic apparatus 100. Alternatively, the display 114 may be a display screen of an electronic device such as a smart phone, a tablet computer, or the like. The number of the displays 114 may be one or more. For example, the display 114 may include a main screen and a touch screen. The main screen may be configured to display ultrasonic images, and the touch screen may be configured for human-machine interaction.

The display 114 may display the ultrasonic image obtained by the processor 112. In addition, the display 114 may provide the user with a graphical interface for human-machine interaction while displaying the ultrasonic images. One or more objects for control may be provided on the graphical interface. The user may use the human-machine interaction device to input operation instructions to control these objects for control, thereby performing corresponding control operations. For example, an icon may be displayed on the graphical interface, which can be operated with the human-machine interaction device to perform specific functions.

Alternatively, the ultrasonic apparatus 100 may include other human-machine interaction devices other than the display 114, which may be communicatively coupled to the processor 112. For example, the processor 112 may be connected to the human-machine interaction device via an external input/output port. The external input/output port may be a wireless communication module, a wired communication module, or a combination thereof. The external input/output port may also be implemented based on USB, bus protocols such as CAN, and/or wired network protocols, etc.

The human-machine interaction device may include an input device configured to detect the input information of the user. The input information may be, for example, control instructions for ultrasonic transmitting/receiving timing, operation instructions for drawing points, lines or boxes, etc. on an ultrasonic image, or other instructions. The input device may include one of a keyboard, a mouse, a roller, a trackball, a mobile input device (such as a mobile device or cell phone with a touch display, etc.), a multi-function knob, etc., or a combination thereof. The human-machine interaction device may also include an output device such as a printer or the like.

It should be understood that the components included in the ultrasonic apparatus 100 shown in FIG. 1 are merely illustrative, and the ultrasonic apparatus 100 may include more or fewer components. The present disclosure is not limited thereto.

An ultrasonic imaging method of an embodiment of the present disclosure will be described below with reference to FIG. 2. FIG. 2 shows a schematic flowchart of an ultrasonic imaging method of an embodiment of the present disclosure. In some embodiments, the ultrasonic imaging method 200 shown in FIG. 2 may be performed by the processor 112 of the ultrasonic apparatus 100 shown in FIG. 1 to obtain a rendered image and display it with the display 114. In other embodiments, the ultrasonic imaging method 200 may be performed by the processor of any other computing device to obtain a rendered image and display it with a display coupled with the processor.

In the method of FIG. 2, the transparency coefficients corresponding to different parts of the outer ovary region can be determined, such that different parts of the outer ovary region are presented in different transparency, thereby increasing the three-dimensional sense of the ovary region. Therefore, a rendering effect with clear positional relationships can be presented to the user, such that the user can clearly see the position, size, and inclusion relationship between the ovaries and follicles, which enhances the visual experience and helps to improve work efficiency and accuracy.

Referring to FIG. 2, the ultrasonic imaging method 200 of an embodiment of the present disclosure may include the following steps.

In step 201, the three-dimensional data to be rendered of the object being examined which contains an ovary and a plurality of follicles in the ovary may be acquired. The acquired three-dimensional data may contain the data representing the whole ovary and follicles of the object being examined. The three-dimensional data may be three-dimensional original data or three-dimensional image data.

In some embodiments, the user may scan the ovarian tissue of the object being examined with the probe of the ultrasonic apparatus to collect the three-dimensional data or four-dimensional data. Specifically, the probe of the ultrasonic apparatus may transmit ultrasonic waves to the ovarian tissue of the object being examined. The ovarian tissue may include an ovary and a plurality of follicles in the ovary. The ultrasonic waves are transmitted into the object being examined. In one embodiment, the probe can be placed at a position corresponding to the ovarian tissue on the body surface of the object being examined. In another embodiment, the intracavitary ultrasonic scanning may be used, in which the ultrasonic probe is moved such that the scanning area of the ultrasonic probe covers the ovarian tissue and then ultrasonic waves are transmitted through the ultrasonic probe into the ovary of the object being examined. The object being examined may be an object including ovarian tissue, such as human organs or human tissue structures. Next, the ultrasonic probe receives echoes of the ultrasonic waves returned from the ovarian tissue of the object be examined, and processes such as a beam-forming and a three-dimensional reconstruction may be performed on the echo signals to obtain a three-dimensional ultrasonic image or a four-dimensional ultrasonic image.

In some embodiments, pre-stored three-dimensional data or four-dimensional data may be acquired from the memory. The user may scan the ovarian tissue of the object being examined with the probe of the ultrasonic apparatus to obtain the three-dimensional data or four-dimensional data. The three-dimensional data or four-dimensional data may be stored in the memory or sent to other device for storage. When subsequent image rendering is required, the other device may acquire the three-dimensional data or four-dimensional data from the memory.

In some embodiments, in the case that the four-dimensional data is obtained, the three-dimensional data at a certain time may be selected from the four-dimensional data as the three-dimensional data to be rendered. The three-dimensional data may be selected manually or automatically. The user can view the four-dimensional data by moving the input device such as a mouse, a trackball or the like and select the three-dimensional data containing the whole ovary and follicles as the three-dimensional data to be rendered. The automatic three-dimensional data selection may be achieved by performing an automatic identification on the four-dimensional data with machine learning or deep learning algorithm to select the three-dimensional data to be rendered.

In some embodiments, the three-dimensional data to be rendered may be selected from the four-dimensional data with an object recognition method. The object recognition method is implemented by directly extracting features from the three-dimensional data at each moment and scoring and classifying the data. This algorithm may mainly include: 1) building a database, where the database includes a large number of three-dimensional data and the corresponding calibration result thereof, where the calibration result is whether it is a standard three-dimensional data to be rendered (such as whether it contains a whole organ structure); 2) identification and positioning of the three-dimensional data to be rendered. The object recognition method may include the object recognition method based on traditional machine learning and the object recognition method based on deep learning.

The object recognition method based on traditional machine learning may extract features from the three-dimensional data, such as local context information, texture information, Harr features, etc., and then integrate the features and input them into a classifier, such as a support vector machine (SVM), an Adaboost, a random forest (Random Forest), etc., to perform a classification scoring by discriminating on the features. After the three-dimensional data at all times in the four-dimensional data are scored, the three-dimensional data with the highest score may be selected as the three-dimensional data to be rendered.

The object recognition method based on deep learning may be realized by neural network. The neural network is classification network, and mainly includes a stack of a convolutional layer, an activation layer, a pooling layer and a full connection layer. The features of the three-dimensional data may be extracted by the convolutional layer. The extracted features may be linearly combined in the full connection layer. Finally, the probability of the current image may be output. After the three-dimensional data at all times in the four-dimensional data are processed, the three-dimensional data with the highest probability may be selected as the three-dimensional data to be rendered. Common classification network may include 3D FCN and so on.

In some embodiments, the three-dimensional data to be rendered may be selected from the four-dimensional data according to an object detection method. The ovary region and the follicle region contained in the three-dimensional data of each moment may be detected by the object detection method, and the three-dimensional data to be rendered may be selected according to the number, volume or the like of the detected ovary region and follicle region. This algorithm may mainly include: 1) building a database, where the database includes a large number of three-dimensional data and the corresponding calibration results thereof, where the calibration results are the bounding boxes of the ovary region and the follicle region in the three-dimensional data; 2) detection and positioning steps of the three-dimensional data to be rendered. The object detection method may include the object detection method based on traditional machine learning and the object detection method based on deep learning.

The object detection method based on traditional machine learning may mainly include the following steps: 1) selecting the region by moving a sliding window with different scales and different length-width ratios; 2) extracting the features based on the image blocks in the region (e.g., Harr feature, HOG feature, etc.); and 3) sending the extracted features into the classifier (e.g., SVM, Adaboost, etc.) for classification to determine the ovary region or the follicle region.

The object detection method based on deep leaning may include the object detection method using candidate regions and deep learning classification, the regression method based on deep learning and the object detection method based on point cloud.

The object detection method using candidate regions and deep learning classification may extract candidate regions and classify the candidate regions based on deep learning, such as performing the classification by transforming the convolutional kernel of R-CNN (Selective Search+CNN+SVM), SPP-Net (ROI Pooling), Fast R-CNN (Selective Search+CNN+ROI), Faster R-CNN (RPN+CNN+ROI), R-FCN, etc. into three-dimensional.

The regression method based on deep learning may mainly include dividing the image into S×S×S grids, detecting object (such as ovary region and follicle region) whose center falls on each grid, and outputting the coordinates (such as center point coordinates and length and width, etc.) of each position of the object and the probability of the category to which the object belongs, which may be realized by, e.g., transforming the convolution kernels of the YOLO series (YOLO V1, YOLO V2, YOLO V3) into three-dimensional, SSD, DenseBox or other algorithms.

The object detection method based on point cloud may include the detection method based on point and the detection method based on voxel. The detection method based on point is a method in which the deep learning model is directly applied on point cloud data. The input is an n×3 point cloud tensor. After the network extracts features from the point cloud data, the final detection result may be obtained by modifying the canonical coordinates according to the features. The network may be, e.g., the Point-RCNN. The detection method based on voxel may divide the point cloud into 3D voxels, which are processed by a three-dimensional CNN, and then use multiple detection heads to detect the position to improve the detection performance. In addition, the detection method based on point and the detection method based on voxel may be combined to combine the three-dimensional voxel convolutional neural network (CNN) with the point-net-based ensemble abstraction techniques, so as to accurately estimate the position of three-dimensional objects (e.g. PV-RCNN, point-voxel RCNN).

With the object detection method above, the position the ovary region, the positions and number of the follicle regions or other information can be obtained. The three-dimensional data with the maximum volume (estimated by the size of the bounding box) of ovary region may be selected from the four-dimensional data. Alternatively, the three-dimensional data may be selected according to the number of follicle regions. The selected three-dimensional data may be the three-dimensional data to be rendered.

In some embodiments, the three-dimensional data to be rendered may be selected from the four-dimensional data by an object segmentation method. The ovary region and the follicle regions contained in the three-dimensional data at each moment may be segmented with the object segmentation method, and the three-dimensional data to be rendered may be selected according to the number, volume or the like of the segmented ovary region and follicle regions. During the segmentation, the object segmentation method may be directly used on the three-dimensional data to segment the ovary region and follicle regions. Alternatively, the three-dimensional data may be split into several two-dimensional data, such as being split radially with the center of the ovary region as the axis or being split in parallel. The segmentation may be performed on the two-dimensional data, and the segmentation results may be reconstructed to obtain the final three-dimensional segmentation result.

Traditional segmentation algorithms include segmentation algorithm based on level set (Level Set), random walk (Random Walker) algorithm, graph cut (Graph Cut) algorithm, Snake algorithm, etc. The object segmentation method may also include object segmentation method based on traditional machine learning and object segmentation method based on deep leaning.

The object segmentation method based on traditional machine learning may mainly include: 1) building a database, where, the database includes a large number of ultrasonic data sets and corresponding calibration results thereof (in the case of directly segmenting the three dimensional data, they are three-dimensional data sets; while in the case of segmenting multiple two-dimensional data contained in the three-dimensional data, they are two-dimensional data sets), and the calibration results are the masks of the ovary region and the follicle regions of the ultrasonic data, that is, the segmentation results; and, 2) segmentation step, in which the ultrasonic data is divided into multiple data blocks (in the case of direct segmentation of three-dimensional data, they are S×S×S size image blocks; while in the case of segmentation of two-dimensional data contained in the three-dimensional data, they are S×S size image blocks), the feature extraction is performed on the data blocks, which may be performed by traditional PCA, LDA, Harr features, texture features, etc., or by deep neural networks (such as Overfeat network), and then the extracted features are classified using cascaded classifiers, such as KNN, SVM, random forest, etc., to determine whether the current data block is ovary region or follicle region. The classification result is used as the marking result of the center point of the current data block. Finally, the segmentation of the entire ultrasonic data may be obtained.

The object segmentation method based on deep leaning may mainly include: 1) building a database where, the database includes a large number of ultrasonic data sets and corresponding calibration results thereof (in the case of directly segmenting the three dimensional data, they are three-dimensional data sets; while in the case of segmenting multiple two-dimensional data contained in the three-dimensional data, they are two-dimensional data sets), and the calibration results are the masks of the ovary region and the follicle regions of the ultrasonic data, that is, the segmentation results; and 2) segmentation of the ovary region and the follicle regions, that is, an end-to-end semantic segmentation algorithm. The input may be an image or a three-dimensional point cloud (in the case of direct segmentation on three-dimensional data). In the case of the input being an image, an output image consistent with the input image in size may be obtained through the stacking of a convolutional layer, a pooling layer, an upsampling or a deconvolutional layer, etc. The output image may be directly segmented to obtain the target organ region. This method is a supervised learning method. Common two-dimensional network includes FCN, U-Net, Mask R-CNN, etc., and three-dimensional segmentation network includes 3D U-Net, 3D FCN, Medical-Net, etc. In the case of the input being the point cloud, the data may be represented as a set of point clouds, expressed as a tensor of n×3, where n represents the number of the point clouds. The input data may be aligned through a transformation matrix obtained by leaning to ensure the invariance of the model to the transformation of the feature space. Feature extraction may be performed on the point cloud data, and the features may be aligned. The global features may be connected in series with the local features of the point clouds previously obtained by leaning. And then, an upsampling or the like may be performed so as to obtain the classification result of each data point that may be the segmentation result, such as PointNet or pointNet++.

In the case that the three-dimensional data is split into several two-dimensional data and the segmentation is performed on the two-dimensional data, the results of the segmentation can be reconstructed to obtain the final three-dimensional segmentation result. The segmentation results may be directly reconstructed to obtain the three-dimensional segmentation result, such as being directly added together, or the maximum pixel value, average value, etc. thereof being taken as the segmentation result. Alternatively, the three-dimensional segmentation result may be obtained by interpolation (suitable for radial sampling).

With the object segmentation methods above, the position of the ovary region, the positions and number of the follicle regions or the like in the three-dimensional data can be obtained. The three-dimensional data in which the volume of the ovary region (estimated by the mask region) is maximum may be selected from the four-dimensional data. Alternatively, the three-dimensional data may be according to the number of the follicles. The selected three-dimensional data may be the three-dimensional data to be rendered.

Referring to FIG. 2, in step 202, a detection may be performed on the three-dimensional data to detect the ovary region and the plurality of follicle regions so as to obtain the type (e.g., ovary region or follicle region) and location of the organ region.

In some embodiments, the detection for the ovary region and the follicle region may be performed with an object detection method. The object detection method may detect the ovary region and the follicle regions contained in the three-dimensional data at each moment, thereby obtaining the type and location of the organ regions. The algorithm may mainly include: 1) building a database, where the database includes a large number of three-dimensional data and the corresponding calibration results thereof, where the calibration results are the bounding boxes of the ovary regions and the follicle regions in the three-dimensional data; and 2) detection and positioning steps for the ovary region and the follicle regions. The object detection method may include the object detection method based on traditional machine learning, the object detection method based on deep learning and the object detection method based on point cloud. The specific steps of the algorithms are similar to the object detection methods in step 201, which are described in detail above and will not be repeated here.

In some embodiments, the detection of the ovary region and the follicle regions may be performed with the object segmentation methods. Similar to the object segmentation methods in step 201, when segmenting, the object segmentation method may be used directly on the three-dimensional data to segment the ovary region and follicle regions contained in the three-dimensional data. Alternatively, the three-dimensional data may be split into several two-dimensional data, such as being split radially with the center of the ovary region as the axis or being split in parallel. The segmentation may be performed on the two-dimensional data to obtain several segmentation results, and the several segmentation results may be reconstructed to obtain the final three-dimensional segmentation result. Traditional segmentation algorithms include segmentation algorithm based on level set (Level Set), random walker (Random Walker) algorithm, graph cut (Graph Cut) algorithm, Snake algorithm, etc. The object segmentation method may also include the object segmentation method based on traditional machine learning and the object segmentation method based on deep leaning. The specific steps of the 2D or 3D object segmentation method are similar to the object segmentation methods in step 201, which have been described in detail above and will not be repeated here.

In some embodiments, the user can manually trace the ovary region and the follicle regions, e.g., by moving the mouse, trackball or the like to trace the ovary region and the follicle regions in the three-dimensional ultrasonic image. The processor receives the user's tracing operation and segment the ovary region and the follicle region in the three-dimensional ultrasonic image according to the received tracing operation.

In some embodiments, the object detection method may be performed on the three-dimensional data to detect a part of the ovary region and the plurality of follicle regions and the object segmentation method may be performed on the three-dimensional data to detect the other part of the ovary region and the plurality of follicle regions. That is, several detection algorithms may be used to detect the ovary region and the plurality of follicle regions. For example, the object detection method may be used to detect the ovary region in the three-dimensional data, and the objection segmentation algorithm may be used to detect the plurality of follicle regions in the three-dimensional data. Alternatively, the object detection method may be used to detect the ovary region and one part of the follicle regions in the three-dimensional data, and the object segmentation method may be used to detect the other part of the follicle regions in the three-dimensional data. Alternatively, the object segmentation method may be used to detect the ovary region in the three-dimensional data and the object detection method may be used to detect the plurality of follicle regions in the three-dimensional data.

Referring to FIG. 2, in step 203, the transparency coefficients corresponding to different portions of the ovary region may be determined according to the results of the detection.

In some embodiments, the entire ovary region may be rendered transparently, and each part may have a different transparency coefficient. In these embodiments, it is possible to adaptively determine the corresponding transparency coefficient for each triangular sheet (in the rendering method with surface rendering) or each pixel (in the rendering method with volume rendering) of the entire ovary region. The rules for determining the transparency coefficients may be set in advance. For example, the rule may be that the more perpendicular the normal line of the surface of the ovary region is to the line of sight, the smaller the transparency coefficient is and the more opaque it is. Alternatively, the rule may be that the more perpendicular the normal line of the surface of the ovary region is to a certain direction, the smaller the transparency coefficient and the more opaque it is. Alternatively, the transparency coefficient may be determined according to the angles between the normal line of the surface of the ovary region and different directions.

In some embodiments, the anterior portion, from the user's perspective, of the ovary region may be represented with a variety of different transparency, while other portions of the ovary region may be represented with the same transparency or opacity. The anterior portion of the ovary region may be automatically detected, and the corresponding transparency coefficients may be determined for each triangular sheet or each pixel corresponding to the anterior portion of the ovary region. In these embodiments, the ultrasonic imaging method 200 may further include determining the anterior portion of the ovary region from the user's perspective according to the results of the detection and determining the corresponding transparency coefficients for the anterior portion and other portions, respectively. Specifically, according to the results of the detection, spatial depth of the ovary region or the normal direction of the contour of the ovary region may be obtained. The spatial depth of the ovary region or the normal direction of the contour of the ovary region may be obtained, for example, from the vertex coordinates of the mesh model for constructing the ovary region. After that, according to the spatial depth or the normal direction of the contour, the anterior portion of the ovary region from the user's perspective may be determined, and the corresponding transparency coefficients may be determined for the anterior portion and the other portion respectively. The fixed transparency coefficient can be, for example, 0 (i.e. opaque), or can be a transparency coefficient smaller than that of the anterior portion (i.e. less transparent than the anterior portion). The fixed transparency coefficient can be preset or can be inputted by the user.

In some embodiments, the target portion of the ovary region under the user's perspective (e.g., the left or right half under the user's perspective, or a sub-region) may be represented with a variety of different transparency, while the other portions of the ovary region under the user's perspective may be represented with the same transparency or opacity. The target portion may be determined first, and then the corresponding transparency coefficient may be determined for each triangular sheet or each pixel corresponding to the target portion. The target portion may be determined automatically (e.g., according to the user's perspective). Alternatively, the target portion may be selected by the user. The processor may determine the target portion in response to the user's selection operation. In these embodiments, the ultrasonic imaging method 200 may further include determining the target portion to be rendered transparently in the ovary region, and determining the corresponding transparency coefficient for the target portion and the other portions, respectively. The fixed transparency coefficient can be, for example, 0 (i.e. opaque), or can be a transparency coefficient smaller than that of the target portion (i.e. less transparent than the anterior portion). The fixed transparency coefficient can be preset or can be inputted by the user.

The process of adaptively determining the transparency coefficient for the entire ovary region or a portion thereof will be described below.

In some embodiments, the method of surface rendering may be used to obtain the rendered image. The vertex coordinates of the mesh model of the ovary region may be obtained from the three-dimensional data according to the results of the detection. The vertex coordinates of the mesh model may be used to form a plurality of triangular sheets for constructing the mesh of the ovary region. After the vertex coordinates of the mesh model are obtained, the transparency coefficients corresponding to at least part of the triangular sheets may be determined. Specifically, in the case that the transparency coefficients are adaptively determined for the entire ovary region, the corresponding transparency coefficients may be determined for all triangular sheets of the mesh model of the ovary region; and in the case that the transparency coefficients are adaptively determined only for the anterior portion or the target portion of the ovary region, the corresponding transparency coefficients may be determined for those triangular sheets corresponding to the anterior portion or the target portion in the mesh model of the ovary region.

In one embodiment, the normal vector of the triangular sheet may be calculated according to the coordinates of the three vertices of the triangular sheet, the angle between the normal vector and a preset reference direction may be calculated, and the transparency coefficient corresponding to the triangular sheet may be determined according to the size of the angle.

In one embodiment, the direction vectors between the three vertices of the triangular sheet and the center of gravity of the mesh model may be calculated according to the coordinates of the three vertices of the triangular sheet, the angles between the direction vectors and a preset reference direction may be calculated, and the transparency coefficient corresponding to the triangular sheet may be determined according to the size of the angles.

In one embodiment, the angles between the three vertices of the triangular sheet and a preset reference direction may be calculated according to the coordinates of the three vertices, and the transparency coefficient corresponding to the triangular sheet may be determined according to the size of the angles.

In the embodiments above, the same preset reference direction may be used, such as the direction of the line of sight or a certain fixed direction. Alternatively, different preset reference directions may be used for different triangular sheets. Alternatively, the triangular sheets may be grouped, and one preset reference direction may be used for each group of triangular sheets. In addition, the rules for calculating the transparency coefficient may be set according to the preset reference direction. For example, in the case that the preset reference direction is the direction of the line of sight, the rule may be set as that the larger the angle is, the smaller the transparency coefficient is.

In some embodiments, the method of volume rendering may be used to obtain the rendered image. The transparency coefficients corresponding to at least part of the pixels for drawing the ovary region may be determined. Specifically, in the case that the transparency coefficients are adaptively determined for the entire ovary region, the corresponding transparency coefficients may be determined for all pixels in the ovary region. In the case that the transparency coefficients are adaptively determined only for the anterior portion or the target portion of the ovary region, the corresponding transparency coefficients may be determined for those pixels corresponding to the anterior portion or the target portion.

In one embodiment, according to the results of the detection, the contour of the ovary region may be segmented. And then, the transparency coefficients of at least part of the pixels within the contour of the ovary region may be determined according to the volume rendering algorithm. For example, a ray tracing algorithm may be used. In one example of volume rendering, a plurality of rays passing through the three-dimensional data is emitted based on the direction of the line of sight. Each ray is incremented according to a fixed step size, and the three-dimensional data on the ray is sampled. The opacity of each sampling point may be determined according to the gray value of said sampling point. The opacities of the sampling points on each ray may be accumulated to obtain an accumulated opacity. The accumulated opacity on each ray may be mapped to a transparency coefficient, and then the transparency coefficient may be mapped to a pixel of a two-dimensional image. In this way, the transparency coefficients of the pixels corresponding to all rays may be obtained, thereby obtaining the rendered image with different transparency.

In one embodiment, the mapping relationship between the gray value and the transparency coefficient can be set in advance. The gray value of the pixels within the contour of the ovary region may be extracted from the three-dimensional data, and then the transparency coefficients of the pixels may be determined according to the mapping relationship.

In step 204, the three-dimensional data may be rendered according to the determined transparency coefficients to obtain the rendered image of the ovary region and the plurality of follicle regions. In this rendered image, different portions of the ovary region are represented with different transparency.

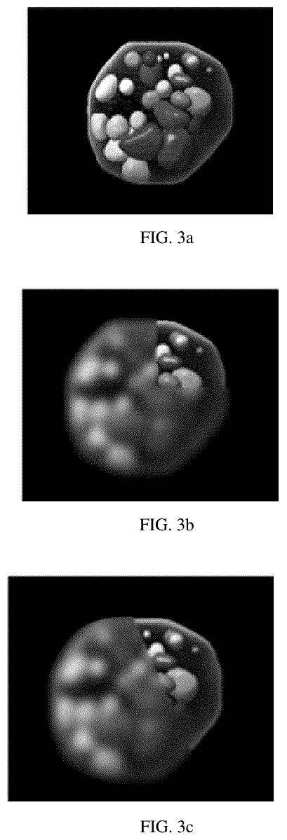

In some embodiments, the entire ovary region may be rendered transparently, and the portions thereof may be presented with different transparency. FIG. 3a shows a schematic diagram. As shown in FIG. 3a, the more perpendicular the portion of the entire ovary region is to the line of sight (e.g., the edge portion), the lower the transparency is, i.e., the more opaque it is; while the smaller the angle between the portion and the user's line of sight (e.g., the portion near the middle of the ovary region) is, the higher the transparency is, i.e., the more transparent it is.

In some embodiments, the anterior portions of the ovary region from the user's perspective may be represented with different transparency, while other portions of the ovary region from the user's perspective may be represented with the same transparency or opacity. In this way, the amount of computation for the transparency coefficients can be reduced without affecting the user's observation, thereby speeding up the rendering process.

In some embodiments, the target portion of the ovary region under the user's perspective may be represented with different transparency, while other portions of the ovary region under the user's perspective may be represented with the same transparency or opacity. FIG. 3b shows a schematic diagram. As shown in FIG. 3b, the upper right portions of the ovary region are represented with a variety of different transparency, where the portions that are more perpendicular to the user's line of sight (e.g., the edge portion) have lower transparency, i.e., are more opaque; while the portions that have smaller angle to the user's line of sight (e.g., the portion near the middle of the ovary region) have higher transparency, i.e., are more transparency. Other portions of the ovary region are represented with the same transparency that are less than that of the upper right portion, i.e., are less transparent than the upper right portions. FIG. 3c shows another schematic diagram. As shown in FIG. 3c, the portion that is represented transparently at the right side of the ovary region is slightly larger than that in FIG. 3b. By adaptively determining the transparency coefficients for the target portion of the ovary region, the calculation amount of the transparency coefficients can be reduced without affecting the user's observation, thereby speeding up the rendering process.

In some embodiments, the ovary region and a plurality of follicle regions may be rendered in any color. The colors for rendering the ovary region and the plurality of follicle regions may be different. In addition, the colors for rendering the follicle regions may also be different so as to distinguish between the follicle regions with different sizes and positions. In one embodiment, the ultrasonic imaging method 200 may further include determining the colors for rendering the ovary region and the plurality of follicle regions. Thereafter, the three-dimensional data may be rendered according to the determined colors. This way, the position and size relationship of the ovary region and the follicle regions can be presented to the user more clearly, thereby further enhancing the user's visual experience.

In some embodiments, in addition to the ovary region, the target follicle region may also be rendered transparently. The transparency coefficients thereof may be determined. The target follicle region may be a follicle region that obscures other follicle regions, or be a follicle region selected as needed. The target follicle region may be determined automatically. Alternatively, the target follicle region may be selected by the user, and the processor may determine the target follicle region in response to the user's selection operation. In the rendered image, different portions of the target follicle region may be represented with different transparency. In one embodiment, the ultrasonic imaging method 200 may further include, according to the results of the detection, determining the target follicle region that obscures one or more other follicle regions under the user's perspective among the plurality of follicle regions, and determining the transparency coefficients corresponding to different portions of the target follicle region. Specifically, the spatial depth or the normal directions of the contours of multiple follicle regions may be obtained according to the results of the detection. The spatial depth or the normal directions of the contours of the follicle region may be obtained, for example, from the vertex coordinates of the mesh model for constructing the follicle region. Then, according to the spatial depth or the normal directions of the contours, the target follicle region that obscures one or more other follicle regions under the user's perspective may be determined from the plurality of follicle regions. After the target follicle region is determined, the transparency coefficients of different portions of the target follicle region may be determined. Similar to the ovary region, the transparency coefficients may also be adaptively determined for the whole or target parts of the target follicle region. For example, it is possible that only the overlapping parts of the target follicle region with other follicle regions (i.e. the occlusion part) are rendered transparently and the transparency coefficients thereof are adaptively determined, while the other parts are not rendered transparently. Specifically, the transparency coefficients may be calculated for each triangular sheet or pixel of the whole or target part of the target follicle region. The process for calculating the transparency coefficients is similar to those described above, and will not be repeated here.

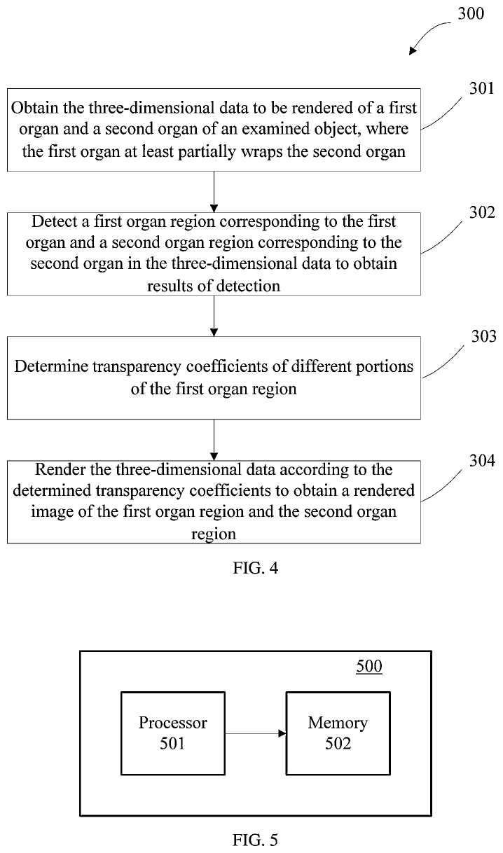

Another ultrasonic imaging method of an embodiment of the present disclosure will be described below with reference to FIG. 4. FIG. 4 shows a schematic flowchart of an ultrasonic imaging method of an embodiment of the present disclosure. In some embodiments, the ultrasonic imaging method 300 in FIG. 4 may be performed by the processor 112 of the ultrasonic apparatus 100 shown in FIG. 1 to obtain the rendered image and display it via the display 114. In other embodiments, the ultrasonic imaging method 300 may be performed by the processor of any other computing device to obtain the rendered image and display it via a display coupled in communication with the processor.

In the method of FIG. 4, when it is desired to render organs between which a wrapping or occlusion relationship exists, the transparency coefficients of the different portions of the outer organ may be determined, such that different portions of the outer organ may be represented with different transparency, which increases the three-dimensional sense of the outer organ. Therefore, a rendering effect with clear positional relationship can be presented to the user, such that the user can clearly see the position, size and inclusion relationship, etc. between the organs, which enhances the visual experience and helps to improve work efficiency and accuracy.

Referring to FIG. 4, the ultrasonic imaging method 300 of an embodiment of the present disclosure may include the following step.

In step 301, the three-dimensional data to be rendered of a first organ and a second organ of the examined object, where the first organ at least partially wraps the second organ. The first organ and the second organ may be any organs or tissues between which a wrapping relationship exists. For example, the first organ may be the ovary, and the second organ may be the follicle. Alternatively, the first organ may be the uterine body, and the second organ may be the endometrium. Alternatively, the first organ may be the fetal brain, and the second organ may be the internal anatomical structure of the brain (e.g. cerebellum, transparent septum, thalamus, and/or lateral ventricle, etc.). Alternatively, the first organ may be the fetal abdomen, and the second organ may be the internal anatomical structure of the fetal abdomen (e.g. gastric vesicles, fetal heart, spine, kidneys, and/or blood vessels, etc.). Alternatively, the first organ may be the liver, and the second organ may be the intrahepatic blood vessels. Alternatively, the first organ may be the heart, and the second organ may be the internal anatomical structure of the heart (e.g., the internal chambers and/or vessels, etc.). The various combinations of the first organ and the second organ described above are merely examples. Any other combinations of the first and second organs that have similar wrapping relationship should also fall within the scope of protection of the present disclosure. The obtained three-dimensional data contains the complete first and second organs of the examined object. The three-dimensional data may be three-dimensional original data or three-dimensional image data.

Similar to the description above with respect to FIG. 2, the three-dimensional data or four-dimensional data may be directly obtained by the user with the probe of the ultrasonic apparatus. Alternatively, it is possible to obtain the pre-stored three-dimensional data or four-dimensional data from the memory. In the case that obtained data is the four-dimensional data, the three-dimensional data at one moment may be selected from the four-dimensional data manually or automatically. This three-dimensional data may be the three-dimensional ultrasonic data with the best quality in the four-dimensional data.

The three-dimensional data may be selected using an object recognition method, an object detection method or an object segmentation method. In some embodiments, the features may be extracted from the three-dimensional data at each moment by the object recognition method, and then the data may be scored and classified according to the features. After the three-dimensional data at all moments in the four-dimensional data are scored, the three-dimensional data with the highest score may be selected as the three-dimensional data to be rendered. In some embodiments, the ovary region and the follicle regions contained in the three-dimensional data at each moment may be detected by the object detection method, and the three-dimensional data to be rendered may be selected according to the number, volume, etc. of the detected ovary region and follicle regions. In some embodiments, the ovary region and follicle regions contained in the three-dimensional data at each moment may be segmented by the object segmentation method, and the three-dimensional data to be rendered may be selected according to the number, volume, etc. of the segmented ovary region and follicle regions. The specific selection process and the algorithms are similar to those described in FIG. 2, and will not be repeated here.

In step 302, the first organ region corresponding to the first organ and the second organ region corresponding to the second organ may be detected in the three-dimensional data to obtain the results of the detection. The results of the detection may include the type (e.g., the first organ or the second organ) and position of the organ regions.

The first organ region and the second organ region may be detected using the object detection method or the object segmentation method. In some embodiments, the first organ region and the second organ region contained in the three-dimensional data of each moment may be detected by the object detection method, thereby obtaining the type and position of the organ regions. In some embodiments, the first organ region and the second organ region contained in the three-dimensional data may be segmented by the object segmentation method (directly segmented, or segmented in the two-dimensional section and then reconstructed), thereby obtaining the type and position of the organ regions. In some embodiments, the user can manually trace the first organ region and the second organ region. The processor may receive the tracing operation of the user and segment the first organ region and the second organ region in the three-dimensional data according to the tracing operation. In some embodiments, one of the first organ region and the second organ region may be detected in the three-dimensional data with the object detection method, while the other of the first organ region and the second organ region may be detected in the three-dimensional data with the object segmentation method. For example, the first organ region located outside in the three-dimensional data may be detected with the object detection method, and the second organ region located inside in the three-dimensional data may be detected with the object segmentation method. The specific detection process and the algorithms may be similar to those described in FIG. 2 and will not be repeated here.

In step 303, based on the results of the detection, the transparency coefficients of different portions of the first organ region may be determined. Based on the results of the detection, the first organ region may be automatically determined as the outside organ, that is, the organ that is desired to be rendered transparently, and the transparency coefficients of different portions thereof may be determined. Alternatively, the user may select the first organ region. The processor may determine the first organ region as the organ that is desired to be rendered transparently in response to the user's selection operation and determine the transparency coefficients of different portions thereof.

In some embodiments, the entire first organ region may be rendered transparently, and the portions thereof may have different transparency coefficients. In these embodiments, it is possible to adaptively determine the transparency coefficients for each triangular sheet (in the case that the method of surface rendering is used) or each pixel (in the case that the method of volume rendering is used) of the entire first organ region. The rules for determining the transparency coefficients may be set in advance. For example, the rule may be that the more perpendicular the normal line of the surface of the first organ region is to the line of sight, the smaller the transparency coefficient is, and the more opaque it is. Alternatively, the rule may be that the more perpendicular the normal line of the surface of the first organ region is to a certain direction, the smaller the transparency coefficient is, and the more opaque it is. Alternatively, the angles between the normal line of the surface of the first organ region and different directions may be calculated to determine the transparency coefficients

In some embodiments, the anterior portion, from the user's perspective, of the first organ region may be represented with a variety of different transparency, while other portions of the first organ region may be represented with the same transparency or opacity. In these embodiments, the anterior portion of the first organ region may be automatically detected, and the transparency coefficients may be determined for each triangular sheet or each pixel of the anterior portion of the ovary region. In these embodiments, the ultrasonic imaging method 300 may further include determining the anterior portion of the first organ region from the user's perspective according to the results of the detection and determining the corresponding transparency coefficients for the anterior portion and other portions, respectively. Specifically, according to the results of the detection, spatial depth of the first organ region or the normal direction of the contour of the first organ region may be obtained. The spatial depth of the first organ region or the normal direction of the contour of the first organ region may be obtained, for example, from the vertex coordinates of the mesh model for constructing the first organ region. After that, according to the spatial depth or the normal direction of the contour, the anterior portion of the first organ region from the user's perspective may be determined, and the corresponding transparency coefficients may be determined for the anterior portion and the other portion respectively. The fixed transparency coefficient can be, for example, 0 (i.e. opaque), or can be a transparency coefficient smaller than that of the anterior portion (i.e. less transparent than the anterior portion). The fixed transparency coefficient can be preset or can be inputted by the user.

In some embodiments, the target portion of the first organ region under the user's perspective (e.g., the left or right half under the user's perspective, or a sub-region) may be represented with a variety of different transparency, while the other portions of the first organ region under the user's perspective may be represented with the same transparency or opacity. The target portion to be rendered transparently of the first organ region may be determined first, and then the corresponding transparency coefficient may be determined for each triangular sheet or each pixel of the target portion of the first organ region. The target portion may be determined automatically (e.g., the portion wrapping the second organ region under the user's perspective). Alternatively, the target portion may be selected by the user. The processor may determine the target portion in response to the user's selection operation. In these embodiments, the ultrasonic imaging method 300 may further include determining the target portion to be rendered transparently in the first organ region, and determining the corresponding transparency coefficients for the target portion and the other portions, respectively. The fixed transparency coefficient can be, for example, 0 (i.e. opaque), or can be a transparency coefficient smaller than that of the target portion (i.e. less transparent than anterior portion). The fixed transparency coefficient can be preset or can be inputted by the user.

The process of adaptively determining the transparency coefficient for the entire first organ region or a portion thereof will be described below.

In some embodiments, the method of surface rendering may be used to obtain the rendered image. The vertex coordinates of the mesh model of the first organ region may be obtained from the three-dimensional data according to the results of the detection. The vertex coordinates of the mesh model may be used to form a plurality of triangular sheets for constructing the mesh model of the first organ region. After the vertex coordinates of the mesh model are obtained, the transparency coefficients of at least part of the triangular sheets may be determined. Specifically, in the case that the transparency coefficients are adaptively determined for the entire first organ region, the corresponding transparency coefficients may be determined for all triangular sheets of the mesh model of the first organ region; and in the case that the transparency coefficients are adaptively determined only for the anterior portion or the target portion of the first organ region, the corresponding transparency coefficients may be determined for those triangular sheets of the anterior portion or the target portion in the mesh model of the first organ region.

In one embodiment, the normal vector of the triangular sheet may be calculated according to the coordinates of the three vertices of the triangular sheet, the angle between the normal vector and a preset reference direction may be calculated, and the transparency coefficient of the triangular sheet may be determined according to the size of the angle.

In one embodiment, the direction vectors between the three vertices of the triangular sheet and the center of gravity of the mesh model may be calculated according to the coordinates of the three vertices of the triangular sheet, the angles between the direction vectors and a preset reference direction may be calculated, and the transparency coefficient corresponding to the triangular sheet may be determined according to the size of the angles.

In one embodiment, the angles between the three vertices of the triangular sheet and a preset reference direction may be calculated according to the coordinates of the three vertices, and the transparency coefficient corresponding to the triangular sheet may be determined according to the size of the angles.

In the embodiments above, the same preset reference direction may be used, such as the direction of the line of sight or a certain fixed direction. Alternatively, different preset reference directions may be used for different triangular sheets. Alternatively, the triangular sheets may be grouped, and one preset reference direction may be used for each group of triangular sheets. In addition, the rules for calculating the transparency coefficient may be set according to the preset reference direction. For example, in the case that the preset reference direction is the direction of the line of sight, the rule may be set as that the larger the angle is, the smaller the transparency coefficient is.

In some embodiments, the method of volume rendering may be used to obtain the rendered image. The transparency coefficients of at least part of the pixels for drawing the first organ region may be determined. Specifically, in the case that the transparency coefficients are adaptively determined for the entire first organ region, the transparency coefficients may be determined for all pixels in the first organ region. In the case that the transparency coefficients are adaptively determined only for the anterior portion or the target portion of the first organ region, the transparency coefficients may be determined for those pixels corresponding to the anterior portion or the target portion.

In one embodiment, according to the results of the detection, the contour of the first organ region may be segmented. And then, the transparency coefficients of at least part of the pixels within the contour of the first organ region may be determined according to the volume rendering algorithm. For example, a ray tracing algorithm may be used. In one example of volume rendering, a plurality of rays passing through the three-dimensional data are emitted based on the direction of the line of sight. Each ray is incremented according to a fixed step size, and the three-dimensional data on the ray is sampled. The opacity of each sampling point may be determined according to the gray value of said sampling point. The opacities of the sampling points on each ray may be accumulated to obtain an accumulated opacity. The accumulated opacity on each ray may be mapped to a transparency coefficient, and then the transparency coefficient may be mapped to a pixel of a two-dimensional image. In this way, the transparency coefficients of the pixels corresponding to all rays may be obtained, thereby obtaining the rendered image with different transparency.

In one embodiment, the mapping relationship between the gray value and the transparency coefficient can be set in advance. The gray value of the pixels within the contour of the first organ region may be extracted from the three-dimensional data, and then the transparency coefficients of the pixels may be determined according to the mapping relationship.

In step 304, the three-dimensional data may be rendered according to the determined transparency coefficients to obtain the rendered image of the first organ region and the second organ region. In this rendered image, different portions of the first organ region are represented with different transparency.

As described above, the entire first organ region may be rendered transparently, and the portions thereof may be represented with different transparency. Alternatively, the anterior portions of the first organ region under the user's perspective may be represented with different transparency, while the other portions of the first organ region under the user's perspective may be represented with the same transparency or opacity. Alternatively, the target portion of the first organ region under the user's perspective may be represented with different transparency, while the other portions of the ovary region under the user's perspective may be represented with the same transparency or opacity.

The present disclosure also provides a computing device. FIG. 5 shows a schematic diagram of a computing device of one embodiment of the present disclosure. As shown in FIG. 5, the computing device 500 may include a processor 501 (e.g., a central processing unit (CPU)) and a memory 502 coupled to the processor 501. The memory 502 may be configured to store computer-executable instructions that, when executed, cause the processor 501 to perform the methods in the above embodiments. The processor 501 and the memory 502 may be connected to each other via a bus, and the input/output (I/O) interface may be also connected to the bus. The computing device 500 may further include several components (not shown in FIG. 5) connected to the I/O interface. The components may include, but not limited to, an input unit such as keyboards or mouse, etc., an output unit such as a display of any type or a speaker, etc., a storage unit such as a magnetic disk or an optical disk, etc. and a communication unit such as a network interface card, a modem or a wireless communication transceiver, etc. The communication unit allows the computing device 500 to exchange information/data with other devices via computer networks such as the Internet and/or various telecommunications networks.

Alternatively, the methods described above may be implemented by a computer-readable storage medium. The computer-readable storage medium may store computer-readable program instructions for carrying out the methods in various embodiments of the present disclosure. The computer-readable storage medium may be a tangible device that may hold and store instructions to be executed by an instruction execution device. The computer-readable storage medium may be, but not limited to, an electrical storage device, a magnetic storage device, an optical storage device, an electromagnetic storage device, a semiconductor storage device, or any suitable combination of the foregoing. More specific examples (non-exhaustive) of the computer-readable storage medium may include a portable computer disk, a hard disk, a random access memory (RAM), a read-only memory (ROM), an erasable programmable read-only memory (EPROM or flash memory), a static random access memory (SRAM), a portable compact disk read-only memory (CD-ROM), a digital versatile disk (DVD), a memory stick, a floppy disk, a mechanical coding device, a punch card or protruding structure in groove on which instructions are stored, and any suitable combination thereof. The computer-readable storage medium herein is not to be interpreted as instantaneous signals per se, such as radio waves or other freely propagating electromagnetic waves, electromagnetic waves propagating through waveguides or other transmitting media (e.g., light pulses through fiber optic cables), or electrical signals transmitted through wires.

Therefore, in one embodiment, a computer-readable storage medium is provided. The computer-readable storage medium has computer-executable instructions stored thereon for executing the methods in various embodiments of the present disclosure.

In general, the various exemplary embodiments of the present disclosure may be implemented in hardware or special purpose circuitry, software, firmware, logic, or any combination thereof. Certain parts may be implemented in hardware, while the other parts may be implemented in firmware or software that is executed by a controller, microprocessor, or other computing device. When the embodiments of the present disclosure are illustrated or described as block diagrams, flowcharts or other graphics, it will be understood that the blocks, devices, systems, techniques or methods described herein may be implemented as non-limiting examples in hardware, software, firmware, special purpose circuitry or logic, general purpose hardware or controllers, or other computing devices, or some combination thereof.

The computer-readable program instructions or computer program products for executing the methods of the various embodiments of the present disclosure may also be stored in the cloud, and when it is desired to be called, the user can access the computer-readable program instructions stored in the cloud for executing the method of one embodiment of the present disclosure through the mobile Internet, a fixed network or other network, thereby implementing the technical solutions disclosed in accordance with the various embodiments of the present disclosure.

It should be understood that the devices and methods disclosed in the embodiments of the present disclosure can be implemented in other ways. For example, the devices described above are merely illustrative. For example, the described units are merely defined by logical functions. When being implemented, the units may be defined in other ways. For example, multiple units or components may be combined or integrated into another device, or some features may be ignored or not performed.

In the present disclosure, numerous specific details have been described. However, it will be understood that the embodiments of the present disclosure may be practiced without these specific details. In some embodiments, well-known methods, structures and techniques have not been described in detail so as not to obscure the understanding to this specification.

Similarly, it will be understood that, in order to simplify the present disclosure and aid in understanding one or more aspects of the present disclosure, the features of the present disclosure have been sometimes grouped together into a single embodiment, diagram, or description thereof in the description of the embodiments of the present disclosure. However, the methods of the present disclosure should not be construed as that the present disclosure claims more features than those expressly recited in each claim. Rather, as recited in the claims, the corresponding technical problems can be solved with fewer features than all features of a disclosed single embodiment. Therefore, the claims that follow the specific embodiments are hereby expressly incorporated into the specific embodiments, where each claim is itself regarded as a separate embodiment of the present disclosure.

Those skilled in the art will appreciate that, except mutually exclusive features, all features disclosed in the present disclosure (including the claims, the abstract and the drawings) and all processes or units of any method or device disclosed may be combined in any combination. Unless expressly stated otherwise, each feature disclosed in the present disclosure (including the claims, the abstract and the drawings) may be replaced by an alternative feature that provides the same, equivalent or similar function.

In addition, those skilled in the art will understand that, although some embodiments described herein include certain features, but not other features, included in other embodiments, a combination of features of different embodiments shall fall within the scope of the present disclosure and form different embodiments. For example, in the claims, any one of the claimed embodiments may be used in any combination.

It should also be noted that the embodiments described above are illustrative, but not mean to limit the present disclosure. Alternative embodiments may be made by those skilled in the art without departing from the scope of the appended claims. In the claims, no reference signs located between parentheses shall be construed as limitations to the claims. The present disclosure may be implemented by means of hardware including several elements and a suitably programmed computer. In the claims enumerating several devices, some of these devices may be embodied by the same hardware. The terms such as first, second and third, etc. herein do not indicate any order. These terms may be interpreted as names.

The description above is merely specific embodiments of the present disclosure or illustration of the specific embodiments. However, the scope of the present disclosure is not limited thereto. Any changes or replacements that can be easily thought of by those skilled in the art within the technical scope of the present disclosure should fall within the protection scope of the present disclosure. The protection scope of the present disclosure should be based on the scope of the claims.

What is claimed is:

1. An ultrasonic imaging method, comprising:
obtaining a three-dimensional data to be rendered of an ovary and multiple follicles wrapped in the ovary of an examined object;
detecting an ovary region corresponding to the ovary and multiple follicle regions corresponding to the multiple follicles in the three-dimensional data;
determining transparency coefficients of different portions of the ovary region according to results of the detection; and
rendering the three-dimensional data according to the determined transparency coefficients to obtain a rendered image of the ovary region and the multiple follicle regions, wherein, in the rendered image, the different portions of the ovary region are represented with different transparency.

2. The method of claim 1, further comprising:
determining anterior portions of the ovary region from a user's perspective; wherein
determining the transparency coefficients of different portions of the ovary region comprises: determining the transparency coefficients of the anterior portions and other portions of the ovary region, respectively; and
in the rendered image, the anterior portions of the ovary region are represented with a variety of different transparency, and the other portions are represented with a same transparency or opacity.

3. The method of claim 1, further comprising:
determining target portions to be rendered in the ovary region; wherein
determining the transparency coefficients of different portions of the ovary region comprises: determining the transparency coefficients of the target portions and the other portions of the ovary region, respectively; and
in the rendered image, the target portions of the ovary region are represented with a variety of different transparency, and the other portions are represented with a same transparency or opacity.

4. The method of claim 1, further comprising:
determining colors for rendering the ovary region and the multiple follicle regions; and
rendering the three-dimensional data according to the determined colors.

5. The method of claim 1, further comprising:
determining a target follicle region that obscures one or more other follicle regions under a user's perspective from the multiple follicle regions according to results of the detection; and
determining transparency coefficients of different portions of the target follicle region; wherein,
in the rendered image, the different portions of the target follicle region are represented with different transparency.

6. The method of claim 1, wherein, rendering the three-dimensional data comprises rendering the three-dimensional data with a surface rendering method, and determining the transparency coefficients of different portions of the ovary region according to results of the detection comprises:
obtaining vertex coordinates of a mesh model of the ovary region from the three-dimensional data according to the results of the detection, wherein the vertex coordinates are used to form a plurality of triangular sheets for constructing the mesh model of the ovary region; and
for at least a part of the plurality of triangular sheets, performing following steps:
calculating a normal vector of the triangular sheet according to the vertex coordinates of three vertices of the triangular sheet;
calculating an angle between the normal vector of the triangular sheet and a preset reference direction; and
determining a transparency coefficient of the triangular sheet according to a size of the angle.

7. The method of claim 1, wherein, rendering the three-dimensional data comprises rendering the three-dimensional data with a surface rendering method, and determining the transparency coefficients of different portions of the ovary region according to results of the detection comprises:
obtaining vertex coordinates of a mesh model of the ovary region and a coordinate of center of gravity of the ovary region from the three-dimensional data according to the results of the detection, wherein the vertex coordinates are used to form a plurality of triangular sheets for constructing the mesh model of the ovary region; and
for at least a part of the plurality of triangular sheets, performing following steps:
calculating direction vectors between three vertices of the triangular sheet and the center of gravity of the mesh model according to the vertex coordinates of the three vertices of the triangular sheet;
calculating angles between the direction vectors and a preset reference direction; and
determining a transparency coefficient of the triangular sheet according to sizes of the angles.

8. The method of claim 1, wherein, rendering the three-dimensional data comprises rendering the three-dimensional data with a surface rendering method, and determining the transparency coefficients of different portions of the ovary region according to results of the detection comprises:
obtaining vertex coordinates of a mesh model of the ovary region from the three-dimensional data according to the results of the detection, wherein the vertex coordinates are used to form a plurality of triangular sheets for constructing the mesh model of the ovary region; and
for at least a part of the plurality of triangular sheets, performing following steps:
calculating angles between three vertices of the triangular sheet and a preset reference direction according to the vertex coordinates of the three vertices of the triangular sheet; and
determining a transparency coefficient of the triangular sheet according to sizes of the angles.

9. The method of claim 1, wherein, rendering the three-dimensional data comprises rendering the three-dimensional data with a volume rendering method, and determining the transparency coefficients of different portions of the ovary region according to results of the detection comprises:
determining a contour of the ovary region according to the results of the detection; and
determining transparency coefficients of at least a part of pixels within the contour of the ovary region corresponding to the volume rendering method.

10. The method of claim 1, wherein, rendering the three-dimensional data comprises rendering the three-dimensional data with a volume rendering method, and determining the transparency coefficients of different portions of the ovary region according to the results of the detection comprises:
determining a contour of the ovary region according to the results of the detection;
obtaining gray values of at least a part of pixels within the contour of the ovary region from the three-dimensional data; and
determining transparency coefficients of the at least a part of pixels according to a preset mapping relationship between gray values and transparency coefficients.

11. The method of claim 1, wherein detecting the ovary region corresponding to the ovary and the multiple follicle regions corresponding to the multiple follicles in the three-dimensional data comprises:
detecting the ovary region and the multiple follicle regions in the three-dimensional data with an object detection method; or
detecting the ovary region and the multiple follicle regions in the three-dimensional data with an object segmentation method; or
detecting a part of the ovary region and the multiple follicle regions in the three-dimensional data with an object detection method, and detecting the other part of the ovary region and the multiple follicle regions in the three-dimensional data with an object segmentation method.

12. The method of claim 1, wherein obtaining the three-dimensional data to be rendered of the ovary and the multiple follicles wrapped in the ovary of the examined object comprises:
obtaining a four-dimensional ultrasonic image of the ovary and the multiple follicles of the examined object; and
selecting a three-dimensional ultrasonic image at a moment from the four-dimensional ultrasonic image as the three-dimensional data to be rendered according to one of an object recognition method, an object detection method and an object segmentation method.

13. An ultrasonic imaging method, comprising:
obtaining a three-dimensional data to be rendered of a first organ and a second organ of an examined object, wherein the first organ at least partially wraps the second organ;
detecting a first organ region corresponding to the first organ and a second organ region corresponding to the second organ in the three-dimensional data;
determining transparency coefficients of different portions of the first organ region according to results of the detection; and
rendering the three-dimensional data according to the determined transparency coefficients to obtain a rendered image of the first organ region and the second organ region, wherein, in the rendered image, different portions of the first organ region are represented with different transparency.

14. The method of claim 13, further comprising:
determining anterior portions of the first organ region from a user's perspective according to the results of the detection, wherein
determining the transparency coefficients of different portions of the first organ region comprises: determining the transparency coefficients of the anterior portions and other portions of the first organ region, respectively; and
in the rendered image, the anterior portions of the first organ region are represented with a variety of different transparency, and the other portions are represented with a same transparency or opacity.

15. The method of claim 13, further comprising:
determining target portions to be rendered in the first organ region; wherein
determining the transparency coefficients of different portions of the first organ region comprises: determining the transparency coefficients of the target portions and the other portions of the first organ region, respectively; and
in the rendered image, the target portions of the first organ region are represented with a variety of different transparency, and the other portions are represented with a same transparency or opacity.

16. The method of claim 13, further comprising:
determining colors for rendering the first organ region and the second organ region; and
rendering the three-dimensional data according to the determined colors.

17. The method of claim 13, wherein, rendering the three-dimensional data comprises rendering the three-dimensional data with a surface rendering method, and determining the transparency coefficients of different portions of the first organ region according to the results of the detection comprises:
obtaining vertex coordinates of a mesh model of the first organ region from the three-dimensional data according to the results of the detection, wherein the vertex coordinates are used to form a plurality of triangular sheets for constructing the mesh model of the first organ region; and
for at least a part of the plurality of triangular sheets, performing following steps:
calculating a normal vector of the triangular sheet according to the vertex coordinates of three vertices of the triangular sheet;
calculating an angle between the normal vector of the triangular sheet and a preset reference direction; and
determining a transparency coefficient of the triangular sheet according to a size of the angle.

18. The method of claim 13, wherein, rendering the three-dimensional data comprises rendering the three-dimensional data with a surface rendering method, and determining the transparency coefficients of different portions of the first organ region according to the results of the detection comprises:
obtaining vertex coordinates of a mesh model of the first organ region and a coordinate of center of gravity of the first organ region from the three-dimensional data according to the results of the detection, wherein the vertex coordinates are used to form a plurality of triangular sheets for constructing the mesh model of the first organ region; and
for at least a part of the plurality of triangular sheets, performing following steps:
calculating direction vectors between three vertices of the triangular sheet and the center of gravity of the mesh model according to the vertex coordinates of the three vertices of the triangular sheet;
calculating angles between the direction vectors and a preset reference direction; and
determining a transparency coefficient of the triangular sheet according to sizes of the angles.

19. The method of claim 13, wherein
the first organ is an ovary and the second organ is a follicle; or
the first organ is a uterine body and the second organ is an endometrium; or
the first organ is a fetal brain and the second organ is an internal anatomical structure of the fetal brain; or
the first organ is a fetal abdomen and the second organ is an internal anatomical structure of the fetal abdomen; or
the first organ is a liver and the second organ is an intrahepatic blood vessel; or
the first organ is a heart and the second organ is an internal structure of the heart.

20. An ultrasonic device, comprising:
an ultrasonic probe;
a transmitting/receiving circuit configured to control the ultrasonic probe to transmit ultrasonic waves to an ovary of an object to be examined, and to control the ultrasonic probe to receive echo signals of the ultrasonic waves;
a memory configured to store computer-executable instructions;
a processor configured to execute the computer-executable instructions to:
obtaining a three-dimensional data to be rendered of a first organ and a second organ of an examined object, wherein the first organ at least partially wraps the second organ;
detecting a first organ region corresponding to the first organ and a second organ region corresponding to the second organ in the three-dimensional data;
determining transparency coefficients of different portions of the first organ region according to results of the detection; and
rendering the three-dimensional data according to the determined transparency coefficients to obtain a rendered image of the first organ region and the second organ region, wherein, in the rendered image, different portions of the first organ region are represented with different transparency; and
a display configured to display the rendered image.
